(19) United States
(12) Patent Application Publication
(10) Pub. No.: US 2025/0177466 A1
(43) Pub. Date: Jun. 5, 2025

(54) DIETARY COMPOSITIONS TO EXTEND LONGEVITY WHILE MAXIMIZING STRENGTH AND FUNCTION

(71) Applicants: UNIVERSITY OF SOUTHERN CALIFORNIA, Los Angeles, CA (US); ISTITUTO FONDAZIONE DI ONCOLOGIA MOLECOLARE ETS, Milan (IT)

(72) Inventors: Valter D. LONGO, Los Angeles, CA (US); Maura FANTI, Los Angeles, CA (US); Maira DI TANO, Milan (IT); Giulia SALVADORI, Milan (IT)

(73) Assignees: UNIVERSITY OF SOUTHERN CALIFORNIA, Los Angeles, CA (US); ISTITUTO FONDAZIONE DI ONCOLOGIA MOLECOLARE ETS, Milan (IT)

(21) Appl. No.: 18/287,381

(22) PCT Filed: Aug. 7, 2023
(86) PCT No.: PCT/US2023/029604

§ 371 Date: Oct. 18, 2023

Related U.S. Application Data
(60) Provisional application No. 63/400,090, filed on Aug. 23, 2022.

(51) Int. Cl. A61K 36/48; A23L 33/00; A61K 31/05; A61K 31/195; A61K 31/198; A61K 31/718; A61K 33/10; A61K 33/42; A61K 36/21; A61K 36/23; A61K 36/31; A61K 36/42; A61K 36/53; A61K 36/63; A61K 36/81; A61K 36/88; A61P 43/00

(52) U.S. Cl. CPC A61K 36/48; A23L 33/40; A61K 31/05; A61K 31/195; A61K 31/198; A61K 31/718; A61K 33/10; A61K 33/42; A61K 36/21; A61K 36/23; A61K 36/31; A61K 36/42; A61K 36/53; A61K 36/63; A61K 36/81; A61K 36/88; A61P 43/00

(57) ABSTRACT

A longevity diet composition includes non-refined complex carbohydrates in an amount from about 45 to 65 percent of the total calories of the longevity diet composition, proteins in an amount from about 7 to 18 percent of the total calories of the total calories of the longevity diet composition, and healthy fats in an amount from about 20 to 40 percent of the total calories of the total calories of the longevity diet composition.

### CROSS-REFERENCE TO RELATED APPLICATIONS

This application claims the benefit of U.S. provisional application Ser. No. 63/400,090 filed Aug. 23, 2022, the disclosure of which is hereby incorporated in its entirety by reference herein.

### STATEMENT REGARDING FEDERALLY SPONSORED RESEARCH OR DEVELOPMENT

This invention was made with government support under grant no. AG055369, awarded by the National Institutes of Health (NIH). The government has certain rights in the invention.”

### TECHNICAL FIELD

In at least one aspect, a nutritional plan for increasing health and lifespan is provided.

### BACKGROUND

Aging is the main risk factor for chronic diseases with a great impact on the social and health care system^{1,2}. Many efforts have been made to delay and reverse the processes associated with aging and related diseases. Among others, calorie restriction (CR) is extremely effective in ameliorating overall health and increasing the lifespan in yeast, multicellular invertebrates, and mouse models and in reducing disease risk factors in humans by acting on molecular pathways involved in nutrient sensing pathways such as insulin/insulin-like growth factor pathways^{3,4}. Therefore, modulating these molecular pathways represents a concrete opportunity to increase longevity and duration of health also in humans. However, CR is not suitable for long-term approaches in humans since it causes a major loss in muscle mass and potential side effects in many systems.

Accordingly, alternative nutritional interventions are needed that: 1) achieve or surpass the effects of CR on longevity, 2) do not require a reduction in calorie intake, 3) do not cause a loss in muscle and bone mass, 4) maintain function and strength higher than what can be achieved with other diets that do not increase longevity.

### SUMMARY

In at least one aspect, a Longevity Diet (LD) composition for improving overall health and lifespan is provided. The LD composition includes non-refined complex carbohydrates in an amount from about 45 to 65 percent of the total calories of the LD composition; proteins in an amount from about 7 to 18 percent of the total calories; and healthy fats in an amount from about 20 to 40 percent of the total calories. Advantageously, the calorie ratio of omega-6 to omega-3 is from 2 to 4.

In another aspect, a Longevity Diet High Strength and Function (LHSF) composition for improving overall health, strength, function, and lifespan is provided. The LHSF composition includes non-refined complex carbohydrates in an amount from about 45 to 65 percent of the total calories of the longevity diet composition; proteins in an amount from about 7 to 18 percent of the total calories; healthy fats in an amount from about 20 to 40 percent of the total calories; and L-methionine supplementation. Typically, the L-methionine supplementation from either pure L-methionine, fish meal, or another plant-based source containing methionine is present in an amount from about 0.01 weight percent to about 1 weight percent of the total weight of the LHSF composition.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIG. 1. Longevity Diet (LD1) followed by Longevity Diet High Strength and Function (LHSF1) increases the lifespan of female and male HET3 mice. Kaplan-Meier plots of survival curves comparing mice treated with LD and WD. Each group consisted of 84 (LD-LHSF) and 82 (WD) mice. Treatments continue until mice died or became moribund. LD-LHFS, Longevity Diet switched to Longevity Diet High Strength and Function (blue); WD, Western Diet (purple).

FIGS. 2A, 2B, 2C, and 2D. LD1 induces rapid weight loss in both females and males HET3 mice. Switching to the LHSF1 diet after 2-4 weeks stabilizes and maintains the body weight. A-C, female absolute and relative body weight; B, D male absolute and relative body weight. The graph shows 6 months of intervention starting at 20 months of age. The red arrows indicate when the switch takes place. HHPD, Healthy High Protein Diet (black); LD-LHSF, Longevity Diet switched to Longevity Diet High Strength and Function (blue); WD, Western Diet (purple).

FIGS. 3A, 3B, 3C, 3D, 3E, 3F, 3G, and 3H. Longevity Diet (LD1) followed by Longevity Diet High Strength and Function (LHSF1) exerts a strong effect on the body composition of both female and male HET3 mice. LD-LHSF intervention significantly reduces body weight (A, E) and fat mass (B, F) while maintaining lean body mass expressed as absolute (C, G) and relative to body weight (D, H) after 3 and 6 months of continuous feeding ad libitum. Fat mass and Lean Mass were analyzed by Nuclear Magnetic Resonance (NMR) using the Minispec LF90 (Bruker). Baseline, 20 months old HET3 mice (white); HHPD, Healthy High Protein Diet (black); LD-LHSF, Longevity Diet switched to Longevity Diet High Strength and Function (blue); WD, Western Diet (purple).

FIGS. 4A, 4B, 4C, 4D, 4E, and 4F. Body composition remodeling improves frailty, muscle strength, and motor coordination of both female and male HET3 mice. Frailty Index performed after 3 and 6 months of continuous feeding ad libitum. B, E) Grip Strength performed after 4 months of continuous feeding ad libitum. C, F) Rotarod performed after 4 months of continuous feeding ad libitum. HHPD, Healthy High Protein Diet (black); LD-LHFS, Longevity Diet1 switched to Longevity Diet High Strength and Function1 (blue); WE, Western Diet (purple).

FIGS. 5A, 5B, and 5C. C57BL/6 male mice were switched from a standard chow to the different diets when they were 16 months old and the new diet continued until death. Longevity Diet (LD) and Longevity High Strength and Function (LHSF) decrease body weight (A) and epididimal fat weight (B). LD in particular causes sever weight loss (20% decrease compared to baseline level) (A). Therefore, at 21 months of age, mice fed with LD were switched to LHSF diet and the switch allowed mice to restore body weight at the same level of mice fed with LHSF from the beginning. Notably mice fed with LHSF after LD or fed with LHSF from the beginning, show an extended survival compared to mice fed with vegan diet and western diet (C).

FIGS. 6A and 6B. Mice fed with LHSF are less frail compared to mice fed with vegan and western diet. LD increases frailty at 21 months (A), however the effect is reversed upon switching to LHSF (B). At 28 months, in fact, mice fed with either LHSFT after LD (LD->LHSF) or fed with LHSF from the beginning show a lower frailty index compared with mice fed with western and vegan diet (B).

FIGS. 7A and 7B. LD and LHSF improve cognitive performance and motor coordination, measured by recognition index and rotarod test in 22 (A) and 28 (B) month old mice.

### DETAILED DESCRIPTION

Reference will now be made in detail to presently preferred compositions, embodiments, and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the invention can be obtained within a range of +/−5% of the indicated value.

As used herein, the term “and/or” means that either all or only one of the elements of said group may be present. For example, “A and/or B” shall mean “only A, or only B, or both A and B.” In the case of “only A,” the term also covers the possibility that B is absent, i.e. “only A, but not B.”

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” and “multiple” as a subset. In a refinement, “one or more” includes “two or more.”

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numeral quantity, in a refinement, the term “less than” includes a lower non-included limit that is 5 percent of the number indicated after “less than.” For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, or 1 percent of the number indicated after “less than.”

The term “non-refined complex carbohydrates” refers to types of carbohydrates that are minimally refined. These types of carbohydrates retain most of their natural fiber and nutritional as compared to completely unrefined carbohydrates. Examples of non-refined complex carbohydrates include but are not limited to, whole grains, vegetables, legumes, fruits, and nuts.

Throughout this application, where publications are referenced, the disclosures of these publications in their entireties are hereby incorporated by reference into this application to more fully describe the state of the art to which this invention pertains.

### Abbreviations:

“LD” means Longevity Diet.

“LHSF” means Longevity Diet High Strength and Function.

In at least one aspect, a Longevity Diet (LD) composition for improving overall health and lifespan is provided. The Longevity Diet, without any calorie or time restriction, has been shown here to be effective in improving lifespan, controlling blood glucose, and remodeling body composition with effects on biomarkers for metabolic dysfunction and aging-related diseases. These effects are attributable to a specific composition providing high nourishment but designed to achieve rapid and strong metabolic effects including fat loss and improved insulin sensitivity, etc. with minimal muscle loss in a short period.

The LD composition includes non-refined complex carbohydrates in an amount from about 45 to 65 percent of the total calories of the LD composition; proteins in an amount from about 7 to 18 percent of the total calories; and fats, and in particular, healthy fats in an amount from about 20 to 40 percent of the total calories. As used herein “healthy fats” include monounsaturated and polyunsaturated fats, including omega-3 and omega-6 fatty acids. In some refinements, the non-refined complex carbohydrates are present in an amount of at least 35 percent, 40 percent, 42 percent, 44 percent, 46 percent, 48 percent, or 50 percent of the total calories of the LD composition and at most 70 percent, 65 percent, 60 percent, 58 percent, 56 percent, 54 percent, or 54 percent of the total calories of the LD composition. In some refinements, the healthy fats are present in an amount of at least 15 percent, 20 percent, 22 percent, 24 percent, 26 percent, 28 percent, or 30 percent of the total calories of the LD composition and at most 45 percent, 40 percent, 38 percent, 36 percent, 34 percent, 32 percent, or 30 percent of the total calories of the LD composition. In some refinements, the proteins are present in an amount of at least 5 percent, 7 percent, 8 percent, 9 percent, 10 percent, 11 percent, or 12 percent of the total calories of the LD composition and at most 22 percent, 20 percent, 19 percent, 18 percent, 17 percent, 16 percent, or 15 percent of the total calories of the LD composition.

In another aspect, fats are optimized, in terms of intake, to provide a balanced calorie ratio of omega-6 to omega-3 from 2:1 to 4:1, and optimally about 2.8:1. In some refinements, fats are optimized, in terms of intake, to provide a balanced calorie ratio of omega-6 to omega-3 of at least 1.5:1, 2:1, 2.1:1, 2.2:1, 2.3:1, 2.4:1, 2.5:1, 2.6:1, 2.7:1, 2.8:1, and 2.9:1. In further refinements, fats are optimized, in terms of intake, to provide a balanced calorie ratio of omega-6 to omega-3 of at most 5:1, 4.5:1, 4:1, 3.9:1, 3.8:1, 3.7:1, 3.6:1, 3.5:1, 3.4:1, 3.3:1, 3.2:1, and 3.1:1.

In a refinement, the healthy fats are at least partially provided by fish meals, fish oil, and olive oil.

In another aspect, the proteins are at least partially provided by legumes and fish meals.

In another aspect, the LD composition includes a carbohydrate component selected from the group consisting of potato starch, chickpeas flour, black beans flour, borlotti beans, cabbage, green beans, leeks, tomatoes, parsley, carrot, celery, onion, peas, potatoes, pumpkins, chard, spinach, zucchini, basil, and combinations thereof, the carbohydrate component formulated to provide non-refined complex carbohydrates in an amount from about 45 to 65 percent of the total calories of the LD composition as set forth above.

In another aspect, the LD composition includes a fat component selected from the group consisting of fish meal, olive oil, fish oil, and combinations thereof. Characteristically, the fat component is formulated to provide fats in an amount from about 20 to 40 percent of the total calories as set forth above.

In another aspect, the LD composition includes a supplement component (i.e., the LD supplement) selected from the group consisting of mineral mix, vitamin mix, calcium phosphate/carbonate, choline bitartrate, TBHQ antioxidant, dicalcium phosphate, and combinations thereof. In a refinement, a supplement component is provided as a powder or liquid including isolates selected from the group consisting of proteins from black beans, chickpeas, borlotti beans, green beans, peas, fish meal, 1-methionine, and combinations thereof. In a further refinement, the supplement component includes pure L-methionine supplementation or L-methionine provided by fish meal, or L-Methionine provided by a plant-based protein source rich in methionine.

In another aspect, the LD composition further includes a liquid carrier. In a refinement, the liquid carrier includes water. In a further refinement, the liquid carrier is the only liquid carrier. In a further refinement, the LD composition is formulated as a powder-based shake. In still a further refinement, the LD composition is formulated as a powder-based shake with the components as set forth above and the balance being water.

In another aspect, a method includes a step of substituting at least one subject's meal per day with the LD composition set forth above for a first predetermined number of days. In a variation, the first predetermined number of days is from 1 to 60 days. In some refinements, the first predetermined number of days at least 1 day, 2 days, 5 days, 10 days, 20 days, or 30 days. In further refinements, the first predetermined number of days at most 100 days, 50 days, 30 days, 20 days, 10 days, or 5 days.

In another aspect, a Longevity Diet High Strength and Function (LHSF) composition for improving overall health, strength, function, and lifespan is provided. The LHSF composition includes non-refined complex carbohydrates in an amount from about 45 to 65 percent of the total calories of the LHSF composition; proteins in an amount from about 7 to 18 percent of the total calories of the LHSF composition; healthy fats in an amount from about 20 to 40 percent of the total calories of the LHSF composition; and L-methionine. Typically, the L-methionine is present in an amount from about 0.01 weight percent to about 1 weight percent of the total weight of the LHSF composition. In some refinements, the non-refined complex carbohydrates are present in an amount of at least 35 percent, 40 percent, 42 percent, 44 percent, 46 percent, 48 percent, or 50 percent of the total calories of the LHSF composition and at most 70 percent, 65 percent, 60 percent, 58 percent, 56 percent, 54 percent, or 54 percent of the total calories of the LHSF composition. In some refinements, the healthy fats are present in an amount of at least 15 percent, 20 percent, 22 percent, 24 percent, 26 percent, 28 percent, or 30 percent of the total calories of the LHSF composition and at most 45 percent, 40 percent, 38 percent, 36 percent, 34 percent, 32 percent, or 30 percent of the total calories of the LHSF composition. In some refinements, the proteins are present in an amount of at least 5 percent, 7 percent, 8 percent, 9 percent, 10 percent, 11 percent, or 12 percent of the total calories of the LHSF composition and at most 22 percent, 20 percent, 19 percent, 18 percent, 17 percent, 16 percent, or 15 percent of the total calories of the LHSF composition. In another aspect, fats are optimized, in terms of intake, to provide a balanced calorie ratio of omega-6 to omega-3 from 2:1 to 4:1, and optimally about 2.8:1.

In a variation of the LHSF composition, fats are optimized, in terms of intake, to provide a balanced calorie ratio of omega-6 to omega-3 of at least 1.5:1, 2:1, 2.1:1, 2.2:1, 2.3:1, 2.4:1, 2.5:1, 2.6:1, 2.7:1, 2.8:1, and 2.9:1. In further refinements, fats are optimized, in terms of intake, to provide a balanced calorie ratio of omega-6 to omega-3 of at most 5:1, 4.5:1, 4:1, 3.9:1, 3.8:1, 3.7:1, 3.6:1, 3.5:1, 3.4:1, 3.3:1, 3.2:1, and 3.1:1.

In another aspect, the healthy fats are at least partially provided by fish meals, fish oil, and olive oil.

In another aspect, the proteins are at least partially provided by legumes and fish meals.

In another aspect, the LHSF composition includes a carbohydrate component selected from the group consisting of potato starch, chickpeas flour, black beans flour, borlotti beans, cabbage, green beans, leeks, tomatoes, parsley, carrot, celery, onion, peas, potatoes, pumpkins, chard, spinach, zucchini, basil, and combinations thereof, the carbohydrate component formulated to provide non-refined complex carbohydrates in an amount from about 45 to 65 percent of the total calories of the longevity diet composition.

In another aspect, the LHSF composition includes a fat component selected from the group consisting of fish meal, olive oil, fish oil, and combinations thereof. Characteristically, the fat component is formulated to provide fats in an amount from about 20 to 40 percent of the total calories.

In another aspect, the LHSF composition includes a LHSF supplement component selected from the group consisting of a mineral mix, a vitamin mix, calcium phosphate/carbonate, choline bitartrate, TBHQ antioxidant, dicalcium phosphate, and combinations thereof. In a refinement, a LHSF supplement component is provided as a powder or liquid including isolates selected from the group consisting of proteins from black beans, chickpeas, borlotti beans, green beans, peas, fish meal, 1-methionine, and combinations thereof. In a further refinement, the LHSF supplement component includes pure L-methionine supplementation or L-methionine provided by fish meal, or L-Methionine provided by a plant-based protein source rich in methionine.

In another aspect, the LHSF composition includes a liquid carrier. In a refinement, the liquid carrier includes water. In a further refinement, the liquid carrier is the only liquid carrier. In a further refinement, the LHSF composition is formulated as a shake, and in particular, a powder-based shake. In still a further refinement, the LHSF composition is formulated as a powder-based shake with the components as set forth above and the balance being water.

In still another aspect, a method includes a step of substituting at least one subject's meal per day with the LHSF composition set forth above for a second predetermined number of days. In a variation, the second predetermined number of days is from 1 to 60 days. In some refinements, the second predetermined number of days at least 1 day, 2 days, 5 days, 10 days, 20 days, or 30 days. In further refinements, the second predetermined number of days at most 100 days, 50 days, 30 days, 20 days, 10 days, or 5 days.

In yet another aspect, a method comprising substituting at least one subject's meal per day with the LD composition set forth above for a first predetermined number of days followed by substituting at least one subject's meal per day with the LHSF composition set forth above for a second predetermined number of days. In a variation, the first predetermined number of days is from 1 to 60 days. In some refinements, the first predetermined number of days at least 1 day, 2 days, 5 days, 10 days, 20 days, or 30 days. In further refinements, the first predetermined number of days at most 100 days, 50 days, 30 days, 20 days, 10 days, or 5 days. In a variation, the second predetermined number of days is from 1 to 60 days. In some refinements, the second predetermined number of days at least 1 day, 2 days, 5 days, 10 days, 20 days, or 30 days. In further refinements, the second predetermined number of days at most 100 days, 50 days, 30 days, 20 days, 10 days, or 5 days.

In still another aspect, a Longevity Diet (LD) composition without protein sources is provided. The LD composition includes non-refined complex carbohydrates in an amount from about 45 to 65 percent of the total calories of the LD composition and healthy fats in an amount from about 20 to 40 percent of the total calories.

Tables 1 and 2 provide formulations for the LD composition and the LHSF compositions. It should be appreciated that practice within plus or minus 30 percent of the values indicated is also contemplated.

TABLE 1
Formulations of Longevity Diet

| | | Longevity Diet | | Longevity Diet |
|---|---|---|---|---|
| | Longevity | High Strength and | Longevity | High Strength and |
| Ingredients | Diet (LD) | Function (LHSF) | Diet (LD) | Function (LHSF) |
| g/kg of diet | Composition 1 | Composition 1 | Composition 2 | Composition 2 |
| Potato Starch | 280.3 | 278.5 | 220 | 209.5 |
| Chickpeas flour | 185 | 185 | 115 | 56 |
| Black beans flour | 126.1 | 126.1 | — | — |
| Borlotti beans | — | — | 197.2 | 197.2 |
| Cabbage | 12.3 | 12.3 | 10 | 10 |
| Green beans | 12.1 | 12.1 | 12.5 | 12.5 |
| Leeks | 11.4 | 11.4 | 14.9 | 14.9 |
| Tomatoes | 20.4 | 20.4 | 8.8 | 8.8 |
| Parsley | 0.3 | 0.3 | 4 | 4 |
| Carrot | 30.8 | 30.8 | 46.2 | 46.2 |
| Celery | 10.9 | 10.9 | 6.9 | 6.9 |
| Onion | 5 | 5 | 5.4 | 5.4 |
| Peas | 21.7 | 21.7 | 32.3 | 32.3 |
| Potatoes | 68.2 | 68.2 | 109.3 | 109.3 |
| Pumpkins | 11.2 | 11.2 | 19.6 | 19.6 |
| Chard | — | — | 6.4 | 6.4 |
| Spinach | 12 | 12 | — | — |
| Zucchini | 14.4 | 14.4 | 14.7 | 14.7 |
| Basil | 2.8 | 2.8 | 2.7 | 2.7 |
| Fish meal | 24.15 | 24.15 | — | 52 |
| Olive oil | 91 | 91 | 110 | 110 |
| Fish oil | 7 | 7 | 10 | 10 |
| L-methionine | — | 1.82 | — | 1.5 |
| Mineral mix | 35 | 35 | 35 | 35 |
| Vitamin mix | 10 | 10 | 10 | 10 |
| Calcium | 5 | 5 | 10 | 7 |
| phosphate/carbonate | | | | |
| Choline bitartrate | 2.5 | 2.5 | — | — |
| TBHQ antioxidant | 0.028 | 0.028 | — | — |
| Dicalcium Phosphate | — | — | — | 19 |
| Total (g) | 999.6 | 999.6 | 1002.4 | 1000.9 |

TABLE 2
Longevity Diet without protein sources

| Ingredients | | | |
|---|---|---|---|
| g/kg of diet | LD and LHSF 1 | LD2 | LHSF 2 |
| Potato starch | 278.5-280 | 220.0 | 209.5 |
| Cabbage | 12.3 | 10 | 10 |
| Leeks | 11.4 | 14.9 | 14.9 |
| Tomatoes | 20.4 | 8.8 | 8.8 |
| Parsley | 0.3 | 4 | 4 |
| Carrot | 30.8 | 46.2 | 46.2 |
| Celery | 10.9 | 6.9 | 6.9 |
| Onion | 5 | 5.4 | 5.4 |
| Potatoes | 68.2 | 109.3 | 109.3 |
| Pumpkins | 11.2 | 19.6 | 19.6 |
| Spinach | 12 | — | — |
| Chard | — | 6.4 | 6.4 |
| Zucchini | 14.4 | 14.7 | 14.7 |
| Basil | 2.8 | 2.7 | 2.7 |
| Olive oil | 91 | 110.0 | 110.0 |
| Fish oil | 7 | 10 | 10 |
| Mineral mix | 35 | 35 | 35 |
| Vitamin mix | 10 | 10 | 10 |
| Calcium phosphate | 5 | 10 | 7 |
| Choline bitartrate | 2.5 | — | — |
| TBHQ antioxidant | 0.028 | — | — |
| Dicalcium Phosphate | — | — | 19 |

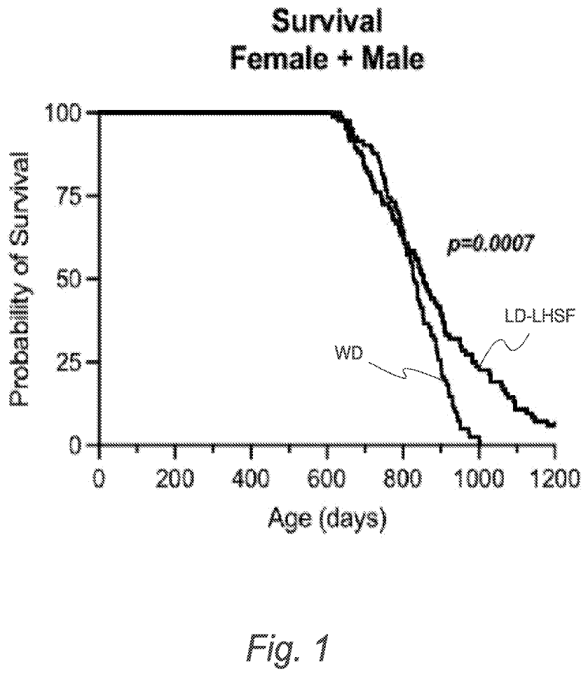

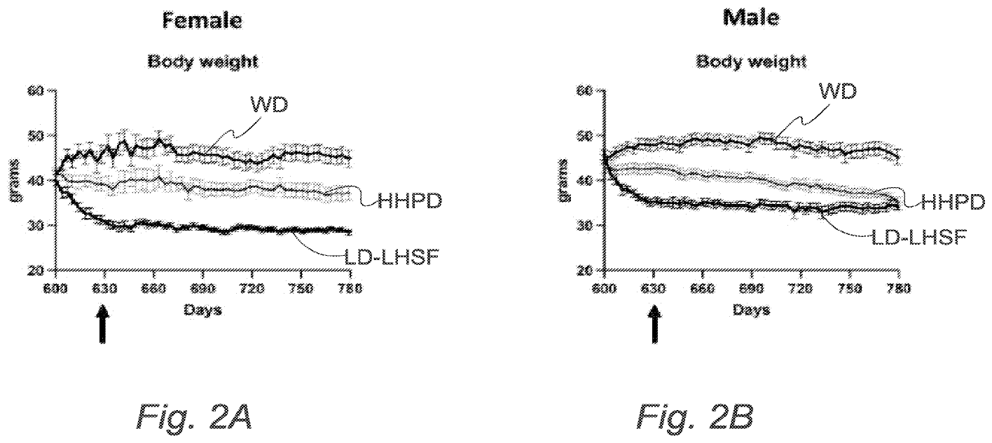

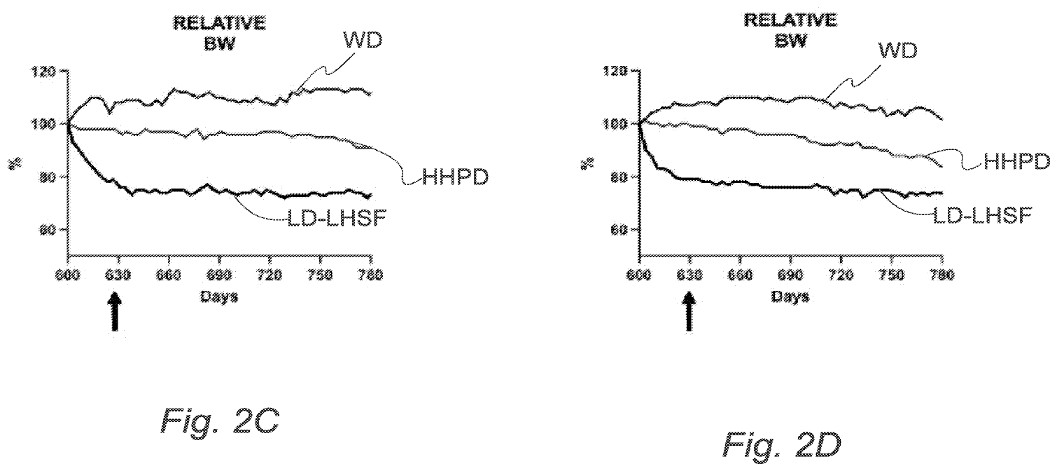

In still another aspect, a Proteins/Amino acid supplement composition that can be used in combination with the compositions of FIG. 1 or 2 is provided. Advantageously, the formulation of the Longevity Diet can be used as a “substitution meal” in combination as needed with “protein source supplementation” to optimize frailty and longevity. Table 3 provides an example of the protein/amino acid supplement composition. It should be appreciated that practice within plus or minus 30 percent of the values indicated is also contemplated.

TABLE 3
Proteins/Amino acid supplementation

| Ingredients | | | | |
|---|---|---|---|---|
| g/kg of diet | LD 1 | LHSF 1 | LD 2 | LHSF 2 |
| Black beans | 126.1 | 126.1 | — | — |
| Chickpeas | 185 | 185 | 115 | 56 |
| Borlotti beans | — | — | 197.2 | 197.2 |
| Green beans | 12.1 | 12.1 | 12.5 | 12.5 |
| Peas | 21.7 | 21.7 | 32.3 | 32.3 |
| Fish meal | 24.15 | 24.15 | — | 52 |
| L-methionine | — | 1.82 | — | 1.5 |
| Plant-based high | — | Providing | — | Providing |
| methionine proteins | | 1.5-2.5 g | | 1.5-2.5 g |
| | | methionine | | methionine |

The following examples illustrate the various embodiments of the present invention. Those skilled in the art will recognize many variations that are within the spirit of the present invention and the scope of the claims.

Studies conducted in our labs on old male C57BL6 mice and both female and male HET3 mice considered the gold standard for aging study, have shown that continuous administration of a new nutritional formulation called “Longevity Diet” (LD) leads to significant improvements in overall health and metabolic markers/risk factors. The Longevity Diet, without any calorie or time restriction, has shown to be effective in controlling blood glucose, and remodeling body composition with effects on biomarkers for metabolic dysfunction and aging-related diseases (see the figures attached). These effects are attributable to the specific composition of the diet which ranges from high fiber and nourishment to low content of proteins and specific amino acids^{8,9}.

The Longevity Diet is a nutritional plan based on plant-based food and a small portion of fish derivates. It provides 58% of its calories from non-refined complex carbohydrates, 12% from proteins provided by legumes and fish meals, and 30% from healthy fats provided by fish meals, fish oil, and olive oil. These last have been optimized, in terms of intake, to provide a balanced ratio of omega-6 to omega-3 around 2.8.

FIG. 1 provides Longevity Diet (LD1) followed by Longevity Diet High Strength and Function (LHSF1) increases the lifespan of female and male HET3 mice. Kaplan-Meier plots of survival curves comparing mice treated with LD and WD. Each group consisted of 84 (LD-LHSF) and 82 (WD) mice. Treatments continue until mice died or became moribund. Longevity Diet switched to Longevity Diet High Strength and Function (LD-LHFS); Western Diet (WD).

FIG. 2 shows that LD1 induces rapid weight loss in both females and males HET3 mice. Switching to the LHSF1 diet after 2-4 weeks stabilizes and maintains the body weight. FIGS. 2A and 2C provide female absolute and relative body weight. Figures B and D male absolute and relative body weight. The graph shows 6 months of intervention starting at 20 months of age. The arrows indicate when the switch takes place. The figures provide plots for a Healthy High Protein Diet (HHPD); LD-LHSF, Longevity Diet switched to Longevity Diet High Strength and Function (blue); and the Western Diet (WD purple).

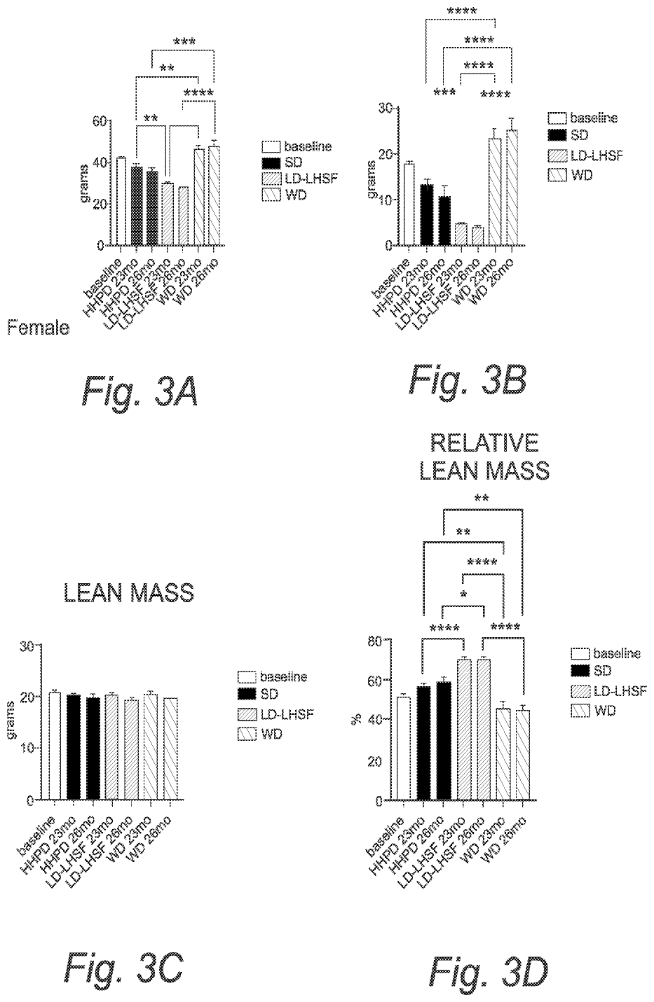

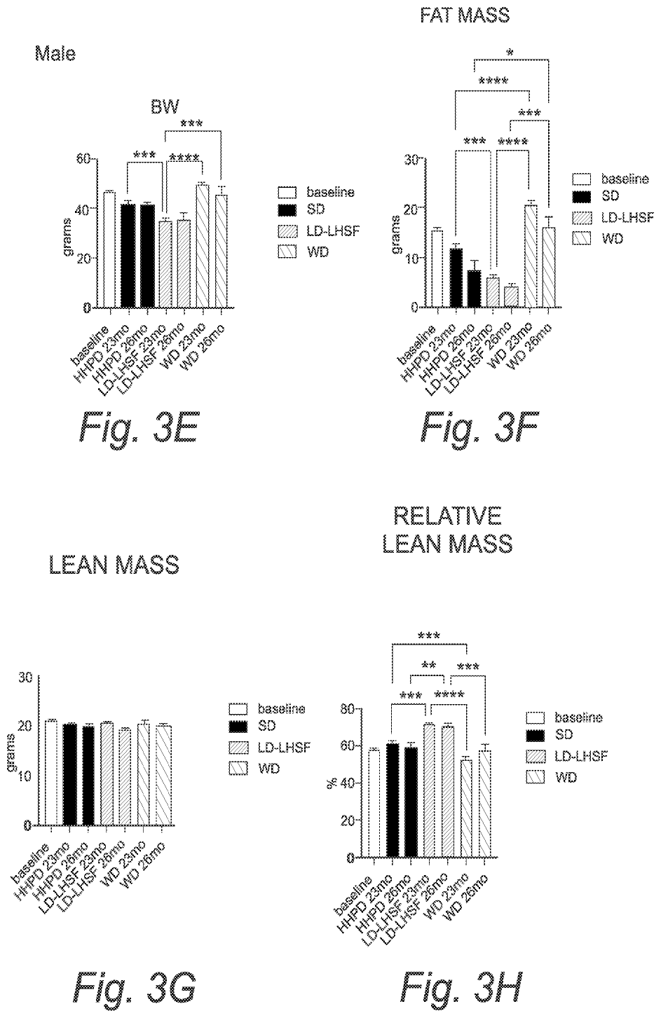

FIG. 3 shows that the Longevity Diet (LD1) followed by Longevity Diet High Strength and Function (LHSF1) exerts a strong effect on the body composition of both female and male HET3 mice. LD-LHSF intervention significantly reduces body weight (A, E) and fat mass (B, F) while maintaining lean body mass expressed as absolute (C, G) and relative to body weight (D,H) after 3 and 6 months of continuous feeding ad libitum. Fat mass and Lean Mass were analyzed by Nuclear Magnetic Resonance (NMR) using the Minispec LF90 (Bruker). Baseline, 20 months old HET3 mice (white); HHPD, Healthy High Protein Diet (black); LD-LHSF, Longevity Diet switched to Longevity Diet High Strength and Function (blue); WD, Western Diet (purple).

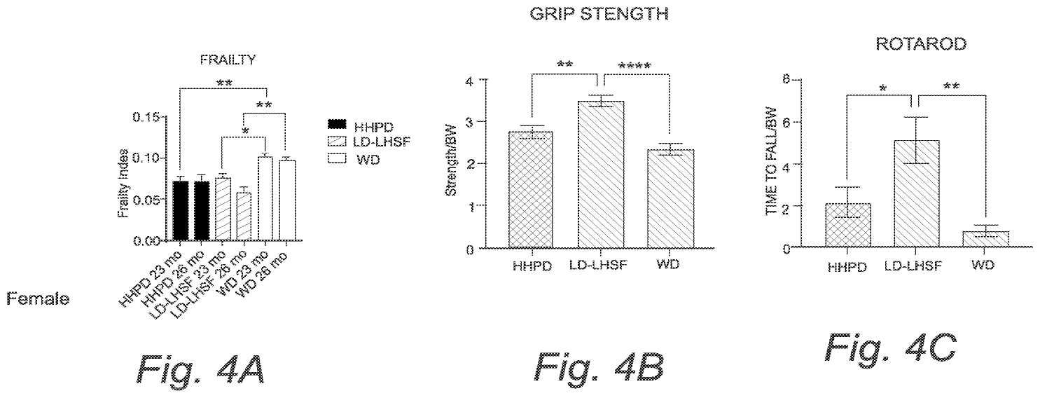

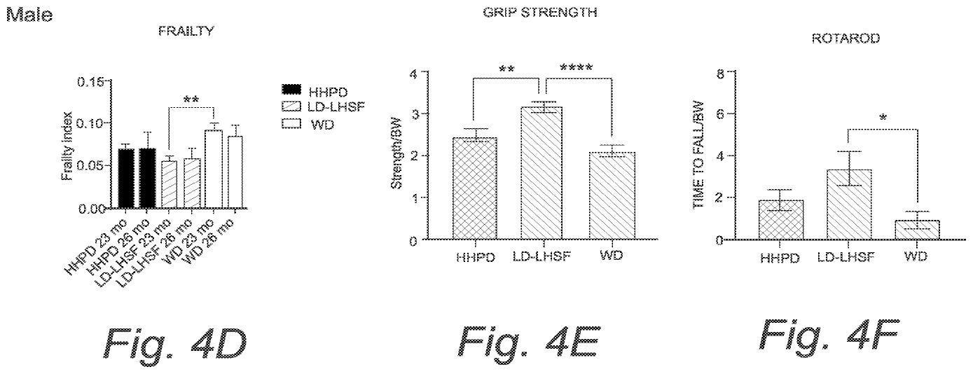

FIG. 4 shows that body composition remodeling improves frailty, muscle strength, and motor coordination of both female and male HET3 mice. Frailty Index performed after 3 and 6 months of continuous feeding ad libitum. FIGS. 4B and 4E shows Grip Strength performed after 4 months of continuous feeding ad libitum. FIGS. 4C and 4F shows results for rotarod performed after 4 months of continuous feeding ad libitum. HHPD, Healthy High Protein Diet (black); LD-LHFS, Longevity Diet1 switched to Longevity Diet High Strength and Function1 (blue); WE, Western Diet (purple).

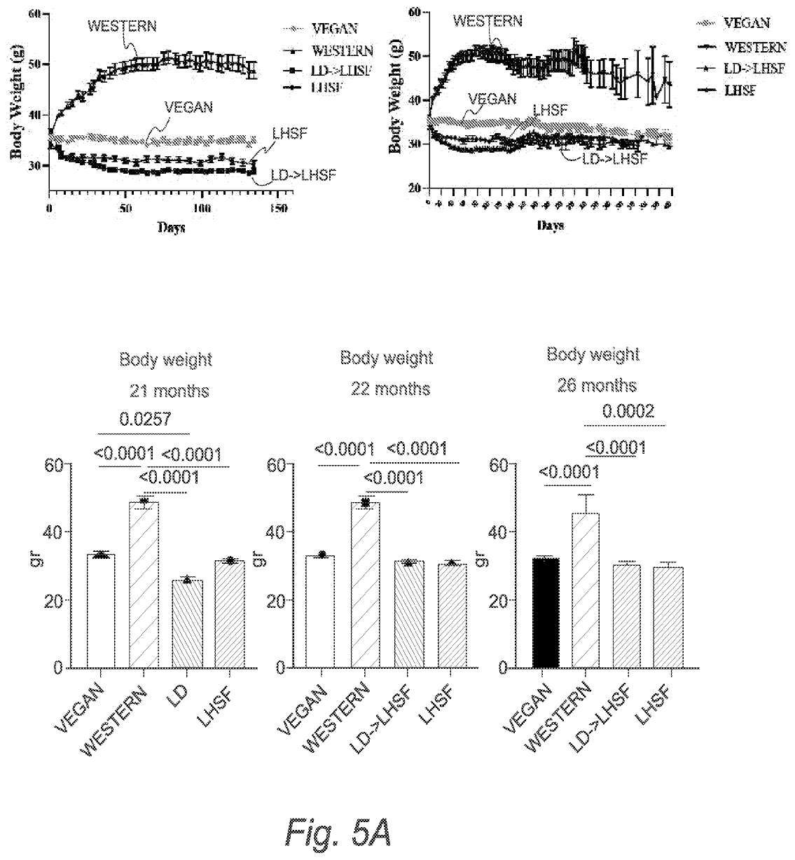

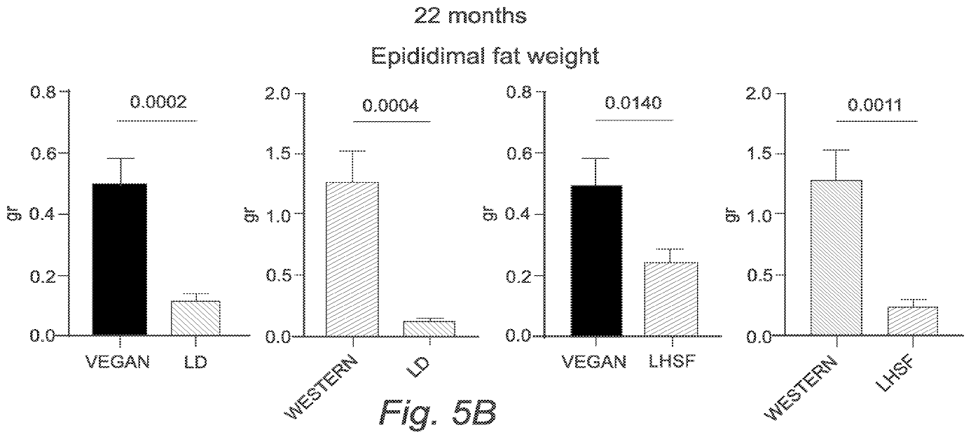

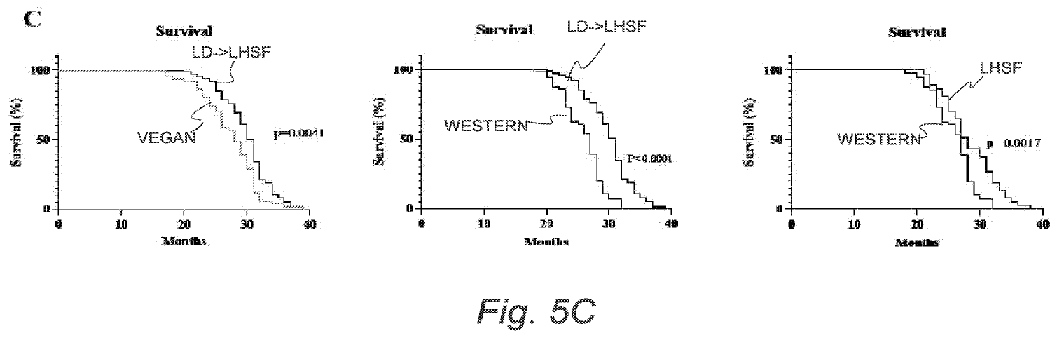

FIG. 5 provides results for when C57BL/6 male mice were switched from a standard chow to the different diets when they were 16 months old and the new diet continued until death. Longevity Diet (LD) and Longevity High Strength and Function (LHSF) decrease body weight (FIG. 5A) and epididimal fat weight (FIG. 5B). LD in particular causes sever weight loss (20% decrease compared to baseline level) (FIG. 5A). Therefore, at 21 months of age, mice fed with LD were switched to LHSF diet and the switch allowed mice to restore body weight at the same level of mice fed with LHSF from the beginning. Notably mice fed with LHSF after LD or fed with LHSF from the beginning, show an extended survival compared to mice fed with vegan diet and western diet (FIG. 5C).

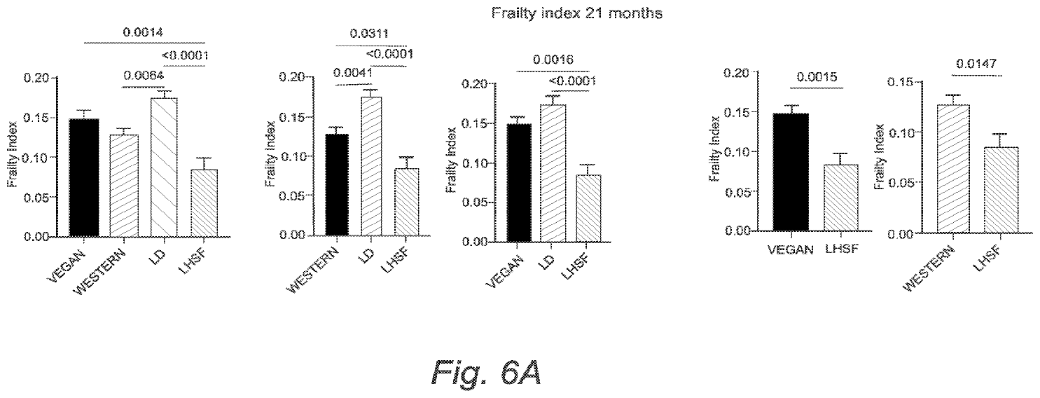

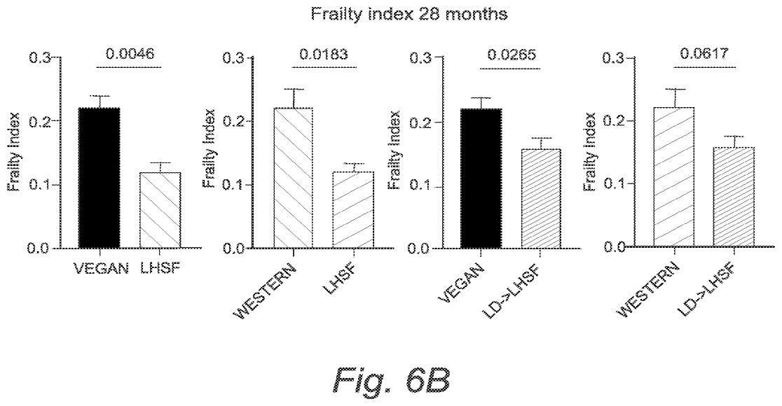

FIG. 6 shows that mice fed with LHSF are less frail compared to mice fed with vegan and western diet. LD increases frailty at 21 months (FIG. 6A), however the effect is reversed upon switching to LHSF (FIG. 6B). At 28 months, in fact, mice fed with either LHSFT after LD (LD->LHSF) or fed with LHSF from the beginning show a lower frailty index compared with mice fed with western and vegan diet (FIG. 6B).

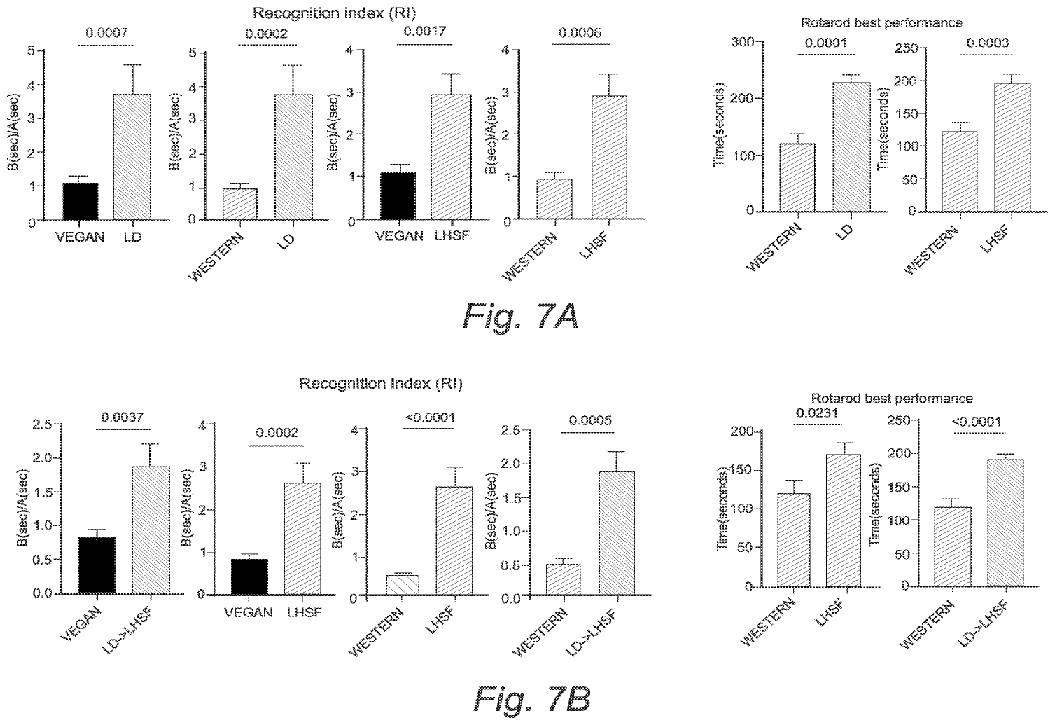

FIG. 7 shows that the LD and LHSF improve cognitive performance and motor coordination as measured by recognition index and rotarod test in 22 (FIG. 7A) and 28 (FIG. 7B) month-old mice.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Niccoli, T. & Partridge, L. Ageing as a Risk Factor for Disease. Current Biology 22, R741-R752 (2012). https://doi.org: 10.1016/j.cub.2012.07.024
2. Mikton, C., de la Fuente-Nunez, V., Officer, A. & Krug, E. Ageism: a social determinant of health that has come of age. Lancet 397, 1333-1334 (2021). https://doi.org: 10.1016/s0140-6736 (21) 00524-9
3. Mattison, J. A. et al. Caloric restriction improves health and survival of rhesus monkeys. Nature Communications 8 (2017). https://doi.org: 10.1038/ncomms14063
4. Fontana, L., Partridge, L. & Longo, V. D. Extending Healthy Life Span-From Yeast to Humans. Science 328, 321-326 (2010). https://doi.org: 10.1126/science.1172539
5. Duregon, E., Pomatto-Watson, L., Bernier, M., Price, N. L. & de Cabo, R.

Intermittent fasting: from calories to time restriction. Geroscience 43, 1083-1092 (2021). https://doi.org: 10.1007/s11357-021-00335-z
6. Brandhorst, S. et al. A Periodic Diet that Mimics Fasting Promotes Multi-System Regeneration, Enhanced Cognitive Performance, and Healthspan. Cell Metabolism 22, 86-99 (2015). https://doi.org: 10.1016/j.cmet.2015.05.012
7. Wei, M. et al. Fasting-mimicking diet and markers/risk factors for aging, diabetes, cancer, and cardiovascular disease. Science Translational Medicine 9 (2017).

https://doi.org: 10.1126/scitranslmed.aai8700
8. Miller, R. A. et al. Methionine-deficient diet extends mouse lifespan, slows immune and lens aging, alters glucose, T4, IGF-I and insulin levels, and increases hepatocyte MIF levels and stress resistance. Aging Cell 4, 119-125 (2005). https://doi.org: 10.1111/j.1474-9726.2005.00152.x
9. Trautman, M. E., Richardson, N. E. & Lamming, D. W. Protein restriction and branched-chain amino acid restriction promote geroprotective shifts in metabolism. Aging Cell 21 (2022). https://doi.org: 10.1111/acel.13626

1. A longevity diet (LD) composition for improving overall health and lifespan comprising:
non-refined complex carbohydrates in an amount from about 45 to 65 percent of the total calories of the LD composition;
proteins in an amount from about 7 to 18 percent of the total calories; and
healthy fats in an amount from about 20 to 40 percent of the total calories wherein a calorie ratio of omega-6 to omega-3 is from 2 to 4.

2. The LD composition of claim 1, wherein the healthy fats are at least partially provided by fish meals, fish oil, and olive oil.

3. The LD composition of claim 1, wherein the proteins are at least partially provided by legumes and fish meals.

4. The LD composition of claim 1 including a carbohydrate component selected from the group consisting of potato starch, chickpeas flour, black beans flour, borlotti beans, cabbage, green beans, leeks, tomatoes, parsley, carrot, celery, onion, peas, potatoes, pumpkins, chard, spinach, zucchini, basil, and combinations thereof, the carbohydrate component formulated to provide the non-refined complex carbohydrates in an amount from about 45 to 65 percent of the total calories of the LD composition.

5. The LD composition of claim 4 including a fat component selected from the group consisting of fish meal, olive oil, fish oil, and combinations thereof, the fat component formulated to provide fats in an amount from about 20 to 40 percent of the total calories.

6. The LD composition of claim 5 including a supplement component selected from the group consisting of a mineral mix, a vitamin mix, calcium phosphate/carbonate, choline bitartrate, TBHQ antioxidant, dicalcium phosphate, and combinations thereof.

7. The LD composition of claim 1 further including a liquid carrier.

8. The LD composition of claim 7, wherein the liquid carrier includes water.

9. The LD composition of claim 8, formulated as a shake.

10. A method comprising substituting at least one subject's meal per day with a longevity diet (LD) composition for a first predetermined number of days, the longevity diet comprising:
longevity diet (LD) composition for improving overall health and lifespan comprising:
non-refined complex carbohydrates in an amount from about 45 to 65 percent of the total calories of the LD composition;
proteins in an amount from about 7 to 18 percent of the total calories; and
healthy fats in an amount from about 20 to 40 percent of the total calories wherein a calorie ratio of omega-6 to omega-3 is from 2 to 4.

11. The LD composition of claim 1, further comprising a longevity diet protein supplement provided as a powder or liquid including isolates selected from the group consisting of proteins from black beans, chickpeas, borlotti beans, green beans, peas, fish meal, 1-methionine, and combinations thereof.

12. A longevity diet high strength and function (LHSF) composition for improving overall health and lifespan comprising:
non-refined complex carbohydrates in an amount from about 45 to 65 percent of the total calories of the longevity diet high strength and function composition;
proteins in an amount from about 7 to 18 percent of the total calories of the longevity diet high strength and function composition;
healthy fats in an amount from about 20 to 40 percent of the total calories of the longevity diet high strength and function composition, wherein a calorie ratio of omega-6 to omega-3 is from 2 to 4; and
L-methionine.

13. The LHSF composition of claim 12, wherein the healthy fats are at least partially provided by fish meals, fish oil, and olive oil.

14. The LHSF composition of claim 12, wherein the proteins are at least partially provided by legumes and fish meals.

15. The LHSF composition of claim 12 including a carbohydrate component selected from the group consisting of potato starch, chickpeas flour, black beans flour, borlotti beans, cabbage, green beans, leeks, tomatoes, parsley, carrot, celery, onion, peas, potatoes, pumpkins, chard, spinach, zucchini, basil, and combinations thereof, the carbohydrate component formulated to provide the non-refined complex carbohydrates in an amount from about 45 to 65 percent of the total calories of the longevity diet high strength and function composition.

16. The LHSF composition of claim 15 including a fat component selected from the group consisting of fish meal, olive oil, fish oil, and combinations thereof, the fat component formulated to provide fats in an amount from about 20 to 40 percent of the total calories.

17. The LHSF composition of claim 16 including a supplement component selected from the group consisting of a mineral mix, a vitamin mix, calcium phosphate/carbonate, choline bitartrate, TBHQ antioxidant, dicalcium phosphate, and combinations thereof.

18. The LHSF composition of claim 12 including a liquid carrier.

19. The LHSF composition of claim 18, wherein the liquid carrier includes water.

20. The LHSF composition of claim 19, formulated as a shake.

21. A method comprising substituting at least one subject's meal per day with a longevity diet high strength and function composition for a predetermined number of days, the longevity diet high strength and function composition comprising:
non-refined complex carbohydrates in an amount from about 45 to 65 percent of the total calories of the longevity diet high strength and function composition;
proteins in an amount from about 7 to 18 percent of the total calories of the longevity diet high strength and function composition;
healthy fats in an amount from about 20 to 40 percent of the total calories of the longevity diet high strength and function composition, wherein a calorie ratio of omega-6 to omega-3 is from 2 to 4; and
L-methionine.

22. A method comprising substituting at least one subject's meal per day with a longevity diet (LD) composition for a first predetermined number of days followed by substituting at least one subject's meal per day with the of the longevity diet high strength and function composition for a second predetermined number of days, the longevity diet comprising:
longevity diet (LD) composition for improving overall health and lifespan comprising:
non-refined complex carbohydrates in an amount from about 45 to 65 percent of the total calories of the LD composition;
proteins in an amount from about 7 to 18 percent of the total calories; and
healthy fats in an amount from about 20 to 40 percent of the total calories wherein a calorie ratio of omega-6 to omega-3 is from 2 to 4; and
the longevity diet high strength and function composition comprising:
non-refined complex carbohydrates in an amount from about 45 to 65 percent of the total calories of the longevity diet high strength and function composition;
proteins in an amount from about 7 to 18 percent of the total calories of the longevity diet high strength and function composition;
healthy fats in an amount from about 20 to 40 percent of the total calories of the longevity diet high strength and function composition, wherein a calorie ratio of omega-6 to omega-3 is from 2 to 4; and
L-methionine.

23. The method of claim 22, wherein the longevity diet further comprises a longevity diet high strength and function protein supplement provided as a powder or liquid including isolates selected from the group consisting of proteins from black beans, chickpeas, borlotti beans, green beans, peas, fish meal, 1-methionine, and combinations thereof.

24. A longevity diet (LD) composition without protein sources for improving overall health and lifespan comprising:
non-refined complex carbohydrates in an amount from about 45 to 65 percent of the total calories of the LD composition; and
healthy fats in an amount from about 20 to 40 percent of the total calories, wherein a calorie ratio of omega-6 to omega-3 is from 2 to 4.

25. The LD composition without protein sources of claim 24, wherein the healthy fats are at least partially provided by fish meals, fish oil, and olive oil.

26. The LD composition without protein sources of claim 24 including a carbohydrate component selected from the group consisting of potato starch, cabbage, leeks, tomatoes, parsley, carrot, celery, onion, potatoes, pumpkins, chard, spinach, zucchini, basil, and combinations thereof, the carbohydrate component formulated to provide the non-refined complex carbohydrates in an amount from about 45 to 65 percent of the total calories of the LD composition.

27. The LD composition without protein sources of claim 26 including a fat component selected from the group consisting of olive oil, fish oil, and combinations thereof, the fat component formulated to provide fats in an amount from about 20 to 40 percent of the total calories.

28. The LD composition without protein sources of claim 27 including a supplement component selected from the group consisting of a mineral mix, a vitamin mix, calcium phosphate/carbonate, choline bitartrate, TBHQ antioxidant, dicalcium phosphate, and combinations thereof.

29. The LD composition without protein sources of claim 28 further including a liquid carrier.

30. The LD composition without protein sources of claim 29, wherein the liquid carrier includes water.

31. The LD composition without protein sources of claim 30, formulated as a shake.

32. A method comprising substituting at least one subject's meal per day with the LD composition without protein sources of claim 1 for a first predetermined number of days.
